(19) United States
(12) Patent Application Publication
(10) Pub. No.: US 2023/0263945 A1
(43) Pub. Date: Aug. 24, 2023

(54) HYDROPHILIC MEDICAL PRODUCTS AND HYDRATION MEDIUMS FOR HYDRATING THE SAME

(71) Applicant: Hollister Incorporated, Libertyville, IL (US)

(72) Inventors: Kevin Murnaghan, Ballina (IE); David J. Farrell, Ballina (IE)

(21) Appl. No.: 18/005,231

(22) PCT Filed: Jul. 13, 2021
(86) PCT No.: PCT/US2021/041460

§ 371 Date: Jan. 12, 2023

Related U.S. Application Data
(60) Provisional application No. 63/050,929, filed on Jul. 13, 2020.

(51) Int. Cl. A61L 29/14; A61M 25/00; A61L 29/16; A61L 29/08

(52) U.S. Cl. CPC A61L 29/14; A61M 25/002; A61M 25/0045; A61L 29/16; A61L 29/08; A61M 2207/10; A61L 2300/204; A61L 2300/404; A61L 2400/10

(57) ABSTRACT

Medical products containing hydration mediums, hydration mediums, and methods of making the same.

The present application claims the benefit of and priority to U.S. Provisional Application No. 63/050,929, filed Jul. 13, 2020, which is hereby incorporated by reference.

### DESCRIPTION

### Technical Field

The present disclosure generally relates to hydrophilic medical device products and hydration mediums for hydrating or wetting such medical devices. More particularly, the present disclosure generally relates to hydrophilic urinary catheter products and hydration mediums for hydrating such hydrophilic urinary catheters.

### Background

Several different devices in different industries are required to be hydrated prior to use and/or stored in a hydrated condition. In certain instances, such devices are stored or packaged in a hydration fluid.

For example, one type of device wherein it may be advantageous to package the device in a hydrated state and/or in a hydration fluid is a hydrophilically coated urinary catheter. In several applications, a coating of hydrophilic material is applied to the surface of the urinary catheter to provide a lubricious surface. When the hydrophilic coating is wetted or hydrated with the hydration fluid, the hydrophilic coating becomes lubricous. The hydration fluid may be, for example, liquid water. The catheter may be placed in a package holding the hydration fluid, such that the catheter is in contact with the hydration fluid.

Typically, the packages containing the catheters and hydration fluid are provided to the user in a sterile state. When the user opens the package for use of the catheter in an intermittent catheter procedure, pathogens including bacteria and microbes, from the environment, may contaminate the hydration medium and/or on the catheter. These pathogens can cause the sterility of the catheter to be compromised. When the catheter is inserted into the urethra of the user, this contamination may increase the risk of the user contracting a urinary tract infection.

Thus, there remains a need for packaged medical products which resist pathogenic contamination of the medical device.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a medical product is disclosed. The product includes a package containing a medical device. The product also includes a hydration medium containing a sulfanilamide.

In another aspect, a hydration medium is disclosed. The hydration medium includes a sulfanilamide.

In another aspect, a method of making a medical product is disclosed. The method comprises placing a medical device within a package and placing a hydration medium into the package. The hydration medium includes a sulfanilamide.

### BRIEF DESCRIPTION OF FIGURES

FIG. 1 is a front plan view of a medical product;

FIG. 2 is a cross-sectional view of the product of FIG. 1; and

FIG. 3 is a perspective view of another embodiment of a medical product that includes a sleeve.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

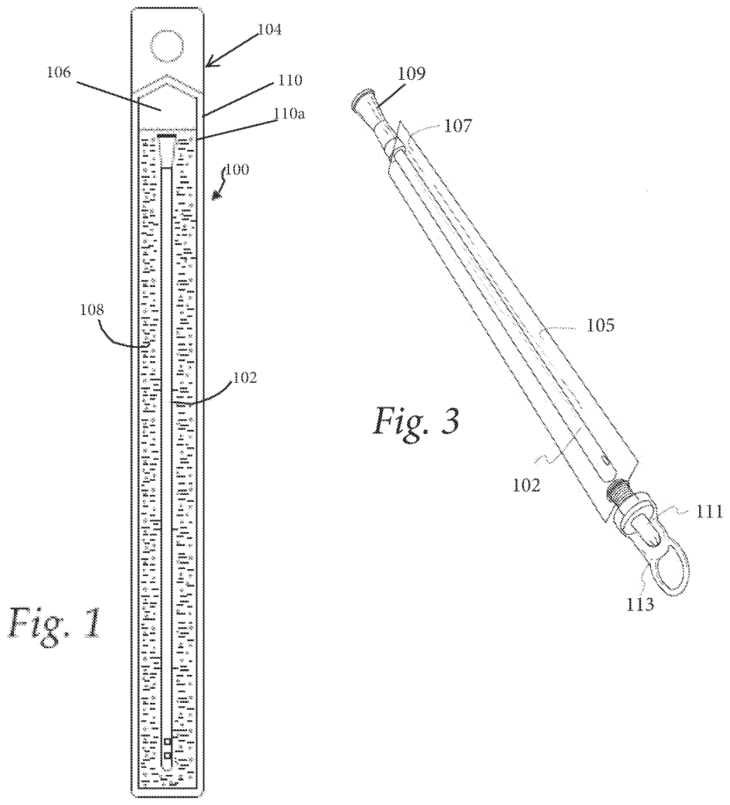

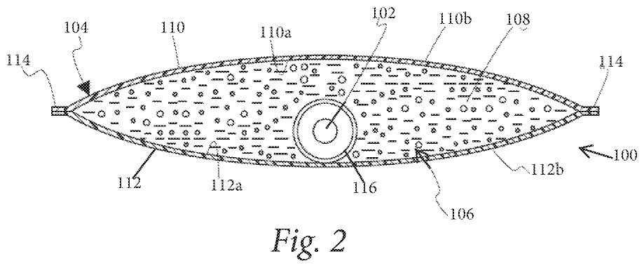

Turning to FIGS. 1-2, these figures illustrate a medical device product 100 comprising a package 104 for containing a medical device 102, such as the illustrated catheter, and a hydration medium 108.

The package 104 may be any suitable package for holding the device 102. For example, the package may be a tear open package, such as a side tear open package, or a peel open package. In the illustrated embodiment, the package 104 includes a front sheet 110 and a back sheet 112 (FIG. 2). The front sheet 110 includes an inner surface 110a facing the back sheet 112, and an outer surface 110b facing the ambient atmosphere. The back sheet 112 includes an inner surface 112a facing the front sheet 110, and an outer surface 112b facing the ambient atmosphere.

The front sheet 110 and back sheet 112 may be made from a liquid and gas impermeable material. For example, the front and back sheets 110 and 112 may be made from a polymer film and/or a metal film. In one embodiment, the material may be a polymer/metal laminate, such as a polymer/aluminum laminate.

The front sheet 110 and back sheet 112 may be sealed to each other to define a cavity 106 for holding the hydration fluid 108. In the illustrated embodiment, the front sheet 110 and back sheet 112 are sealed to each other by a peripheral seal 114. The peripheral seal 114 may be a peelable seal that may be a heat seal, an adhesive seal or any other suitable peelable seal that allows the front sheet 110 to be separated from the back sheet 112 when the sheets are peeled apart during use. As illustrated, the package 104 may include the hydration medium 108 and the medical device 102 inside the package 104. As shown in the embodiment of FIG. 2, the package 104 may also include an inner cavity 116 that contains the medical device 102. The inner cavity 116 may hold the medical device 102 in place while inside the package 104. The inner cavity 116 may allow the hydration medium 108 to contact a surface of the medical device 102 such that the medical device 102 stays hydrated while inside the packaging.

Referring to FIG. 3, the medical device 102 may include a sleeve 105 surrounding or substantially surrounding the medical device 102. For example, when the medical device 102 is a urinary a catheter, the sleeve 105 may surround at least a portion of the catheter. The sleeve 105 may define an internal cavity 107 that contains a portion of the catheter and the hydration medium wherein the hydration medium is contact with the catheter. For example, the hydration medium may be in contact with the hydrophilic portion of the catheter. In the illustrated embodiment, one end of the sleeve 105 is attached at or near the drainage member 109 of the catheter and the other end of the sleeve is attached to an insertion aid 111. The insertion aid 111 may include a cap 113. The medical device 102 including the sleeve 105 may be packaged in any suitable package, such as the packages described herein.

Turning now to the hydration medium 108, the hydration medium 108 may include a sulfanilamide. In one embodiment, the hydration medium may be a fluid for activating the hydrophilic material of the medical device and the sulfanilamide. For example, the hydration medium may include water and the sulfanilamide.

The sulfanilamide may include sulfanilamide, substituted sulfanilamide and sulfanilamide derivatives. The sulfanilamide may be one or more of:

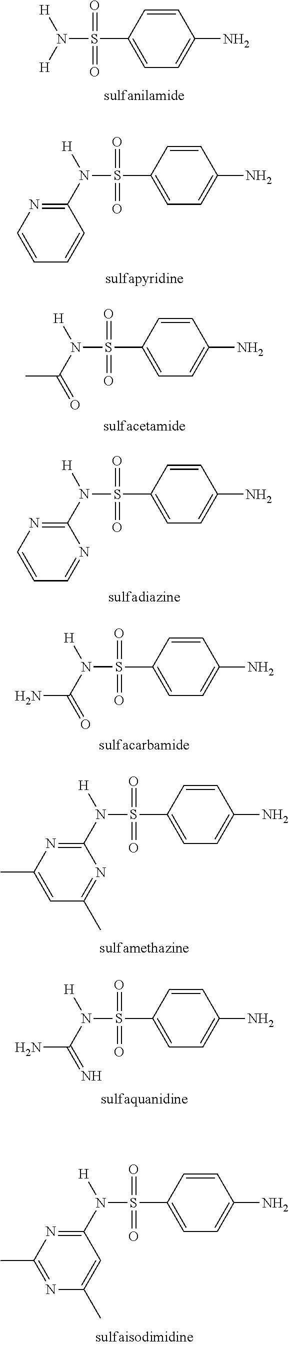

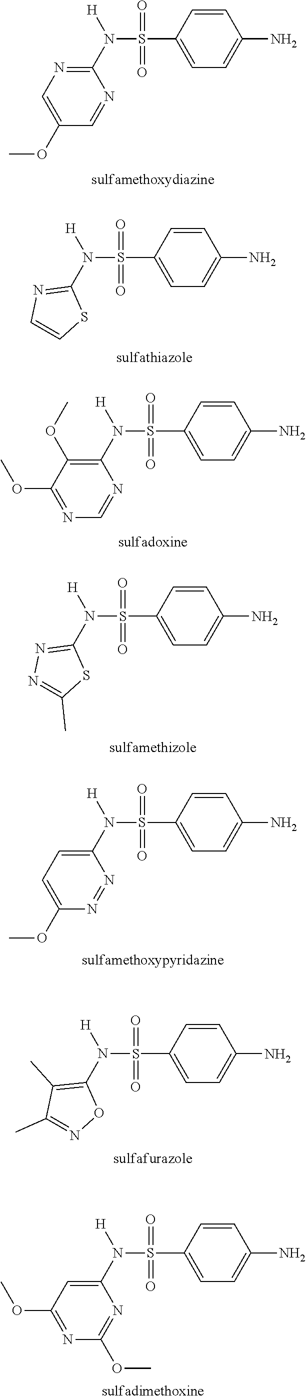

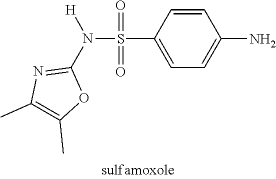 [SMILES: Cc1nc(NS(=O)(=O)c2ccc(N)cc2)oc1C]

The sulfanilamides, substituted sulfanilamides, and sulfanilamide derivatives (“sulfanilamide compounds”) are anti-bacterial and/or anti-microbial. The sulfanilamide compounds may be used to resist pathogens that infect the urinary tract and other areas of the body. For example, both sulfamethoxazole in combination with trimethoprim, and sulfadiazine may be useful in the treatment of urinary tract infections (UTIs). Sulfacetamide and its sodium salt are used in treatments for bacterial eye infections and also as a lotion used for the treatment of acne and dermatitis. Sulfaguanidine is a veterinary anti-bacterial used for intestinal infections. Adding sulfanilamides, substituted sulfanilamides, and sulfanilamide derivatives to the hydration medium 108 may help prevent contamination of the hydration medium 108 and the medical device 102 by inhibiting the proliferation of pathogens such as bacteria, and microbes. Sulfanilamide compounds may be used in sodium salts or in combination with other materials such as sulfamethazole and trimethoprim. However, the hydration medium may include sulfanilamide compounds that are not in salt form.

Additionally, the hydration medium 108 may further include other additive and agents, such as glycerol, antioxidants, osmolality increasing compounds, buffers, foaming agents. For example, in one embodiment the hydration medium may include water in an amount between 98 wt % to 99.9 wt % and sulfanilamides in an amount between 0.01 wt % to 2 wt %. The hydration medium may also include water in an amount between 81 wt % to 99.0 wt % and sulfanilamides in an amount between 0.01 wt % to 2 wt %, and optionally include any or all of the following components: surfactant/foaming agent in an amount between 0.05 wt % to 5 wt %, polyol (such as glycerol) in an amount between 0.5 wt % to 10 wt %, stabilizer (such as Xanthan gum) in an amount between 0.01 wt % to 2 wt %, and hydrogen peroxide in an amount between 0.01 wt % to 0.2 wt %.

The sulfanilamides, substituted sulfanilamides, and sulfanilamide derivatives in the hydration medium 108 may be used at levels between 100-20000 parts-per-million (ppm) within the hydration medium 108.

In an embodiment the hydration medium may comprise sulfanilamide in an amount between about 0.01 wt % (100 ppm) to about 2 wt % (20000 ppm).

In another embodiment the hydration medium may comprise sulfanilamide in an amount between about 0.01 wt % (100 ppm) to about 1 wt % (10000 ppm).

In another embodiment the hydration medium may comprise sulfanilamide in an amount between about 0.02 wt % (200 ppm) to about 1 wt % (10000 ppm).

In another embodiment the hydration medium may comprise sulfanilamide in an amount between about 0.05 wt % (500 ppm) to about 0.1 wt % (1000 ppm). The hydration medium may also comprise glycerol in an amount between about 0.99 wt % (9900 ppm) to about 9.9 wt % (99000 ppm). The sulfanilamide in this embodiment may be sulfacetamide.

In alternative embodiments other suitable levels of the sulfanilamide compounds and/or glycerol may be used.

When the medical device is a catheter, the catheter may include a catheter shaft. The catheter shaft may or may not include a lubricious hydrophilic surface, such as a hydrophilic coating. When a catheter has a hydrophilic coating, the hydration medium may contact the hydrophilic coating to wet or hydrate the hydrophilic coating. When the coating is hydrated, it becomes lubricious. The lubricity eases introduction of the medical device into the body and aids in reducing pain and discomfort associated with such introduction. The hydrophilic coating can be a single layer or a multilayer hydrophilic coating. Multi-layered coating can include at least a base coat and a top layer. In other embodiments that do not include a hydrophilic coating, the medical device may have a lubricious gel on the outer surface of the device. In such an embodiment, the gel may contain the sulfanilamide. The package may also contain a gripping aid, such as a tubular gripper positioned around the catheter shaft. In any of the catheters used, a distal end of the catheter may be connected to a collection bag.

The medical device may be made of any appropriate material known to one of ordinary skill in the art including low-density polyethylene (LDPE), TPE, PU, or other polymeric materials. Other suitable types of hydrophilic medical devices, besides catheters, may be placed in the package.

Additionally, any of the hydration mediums disclosed herein may be used in a foamed or unfoamed state.

In an embodiment, the hydration medium may be foamed to produce a hydration foam or mousse which may be used to hydrate or wet a device or product. The hydration foam may directly contact the hydrophilic coating to hydrate it. The hydration foam includes a mass of gas bubbles formed on or in liquid. Additionally, the creation or formation of the hydration foam may occur at any of the various stages from manufacture to use, depending on the design and use of the medical device. While any of the hydration mediums disclosed herein may be foamed or formed into a foam, depending on the desired use, the hydration mediums disclosed herein also may be employed in an unfoamed state.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A medical product comprising:
a package containing a medical device; and
a hydration medium comprising water and a sulfanilamide.

2. The medical product of claim 1, wherein the hydration medium further comprises a glycerol.

3. The medical product of claim 1, wherein the sulfanilamide is one or more of: sulfapyridine, sulfamethoxydiazene, sulfathiazole, sulfacetamide, sulfadiazine, sulfadoxine, sulfamethizole, sulfacarbamide, sulfamethazine, sulfamethoxypyridazine, sulfamethoxazole, sulfafurazole, sulfaguanidine, sulfaisodimidine, sulfamethoxine, and sulfamoxole.

4. The medical product of claim 1, wherein the sulfanilamide includes sulfanilamide, substituted sulfanilamide, and sulfanilamide derivatives.

5. The medical product of claim 1, wherein the hydration medium comprises sulfanilamide in an amount between about 0.01 wt % to about 2 wt %.

6. The medical product of claim 1, wherein the hydration medium comprises sulfanilamide in an amount between about 0.01 wt % to about 1 wt %.

7. The medical product of claim 1, wherein the hydration medium comprises sulfanilamide in an amount between about 0.02 wt % to about 1 wt %.

8. The medical product of claim 1, wherein the hydration medium comprises sulfanilamide in an amount between about 0.05 wt % to about 0.1 wt %.

9. The medical product of claim 2, the glycerol is in an amount between about 0.99 wt % to about 9.9 wt %.

10. The medical product of claim 1, wherein the medical device is a catheter including a shaft.

11. The medical product of claim 10, wherein the hydration medium contacts the shaft.

12. The medical product of claim 1, wherein the medical device has a hydrophilic coating.

13. The medical product of claim 12, wherein the hydration medium contacts the hydrophilic coating.

14. A hydration medium, comprising:
water; and
a sulfanilamide.

15. The hydration medium of claim 14, further comprising a glycerol.

16. The hydration medium of claim 14, wherein the sulfanilamide is one or more of: sulfapyridine, sulfamethoxydiazene, sulfathiazole, sulfacetamide, sulfadiazine, sulfadoxine, sulfamethizole, sulfacarbamide, sulfamethazine, sulfamethoxypyridazine, sulfamethoxazole, sulfafurazole, sulfaguanidine, sulfaisodimidine, sulfamethoxine, and sulfamoxole.

17. The hydration medium of claim 14, wherein the sulfanilamide includes sulfanilamide, substituted sulfanilamide, and sulfanilamide derivatives.

18. The hydration medium of claim 14, comprising sulfanilamide in an amount between about 0.01 wt % to about 2 wt %.

19. (canceled)

20. (canceled)

21. (canceled)

22. The hydration medium of claim 15, wherein the glycerol is in an amount between about 0.99 wt % to about 9.9 wt %.

23. A method of making a medical product, comprising:
placing a medical device within a package; and
placing a hydration medium into the package, wherein the hydration medium comprises water and a sulfanilamide.
